(19) United States
(12) Patent Application Publication
(10) Pub. No.: US 2022/0186281 A1
(43) Pub. Date: Jun. 16, 2022

(54) METHOD FOR PROCESSING AND ANALYSIS OF VISCOUS LIQUID BIOLOGICAL SAMPLES

(71) Applicant: CEDARS-SINAI MEDICAL CENTER, Los Angeles, CA (US)

(72) Inventors: Gabriela Guimaraes Sousa Leite, Los Angeles, CA (US); Walter Morales, Rowland Heights, CA (US); Stacy Weitsman, Los Angeles, CA (US); Ruchi Mathur, Los Angeles, CA (US); Mark Pimentel, Los Angeles, CA (US)

(73) Assignee: CEDARS-SINAI MEDICAL CENTER, Los Angeles, CA (US)

(21) Appl. No.: 17/425,885

(22) PCT Filed: Jan. 30, 2020
(86) PCT No.: PCT/US20/15939

§ 371 Date: Jul. 26, 2021

Related U.S. Application Data
(60) Provisional application No. 62/798,856, filed on Jan. 30, 2019.

(51) Int. Cl. C12Q 1/24

(52) U.S. Cl. CPC C12Q 1/24

(57) ABSTRACT

The invention provides for methods and kits for processing viscous biological samples such as intestinal fluids and aspirates. Increased numbers of microorganisms are extracted by the methods and kits of this invention, and thus, DNA extraction and analysis can be performed.

### CROSS-REFERENCE TO RELATED APPLICATIONS

This application includes a claim of priority under 35 U.S.C. § 119(e) to U.S. provisional patent application No. 62/798,856 filed Jan. 30, 2019, the entirety of which is hereby incorporated by reference.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The gut microbiome plays important roles in contributing to the health of the human host, and its perturbation has also been linked to a wide variety of diseases and conditions. For example, gut microbes synthesize vitamin, reduce cholesterol and modify bile acids, influence the host immune system and modulate neurohormonal function, regulate epithelial cell proliferation and gut barrier function, break down dietary elements such as starch and fiber and thus provide additional energy sources, and have numerous effects on host metabolism and energy homeostasis, amongst others. As our understanding of the gut microbiome grows, alterations in the balance of gut microbial populations have been linked to diseases and conditions including obesity and diabetes, irritable bowel syndrome, inflammatory bowel disease, non-alcoholic fatty liver disease, and more recently Parkinson's disease, Alzheimer's disease and autism spectrum disorders.

While the studies performed to date have provided invaluable insights into the myriad roles played by the gut microbiome, the vast majority of these have been performed using stool samples, which are not representative of the entire length of the gastrointestinal tract. The human gastrointestinal tract is typically around 25 feet in length, of which the small intestine comprises an average of 20 feet. Conditions such as acidity and transit time vary tremendously along this length, and as a result the microbial populations present as well as overall microbial density also differ significantly, with the small intestine being more acidic and having lower numbers of microbes than the colon. The small intestine, which is divided into the duodenum, ileum and jejunum, is of central importance to the processes of digestion and nutrient absorption, particularly the duodenum which is the site of convergence of chyme from the stomach, enzymes from the pancreas and bile salts from the gall bladder. Moreover, perturbations of small intestinal microbial populations, such as occur in small intestinal bacterial overgrowth (SIBO), are associated with gastrointestinal symptoms including nausea, vomiting, bloating, flatulence, abdominal pain and distension, and diarrhea and/or constipation. Clearly, characterizing the microbial populations of the small intestine is of central importance, but efforts to date have been hampered both by the difficulty of obtaining samples, and by difficulties associated with adapting sample processing and DNA isolation techniques that were designed for stool so that they can be used for small intestinal samples.

Small intestinal samples are typically obtained during an upper endoscopy or esophagogastroduodenoscopy, by passing a catheter through the biopsy channel of the endoscope into the desired intestinal segment (most commonly the duodenum), and using gentle suction to aspirate a small sample of luminal fluid. Care is taken to prevent cross-contamination of samples with secretions from the mouth and stomach, and the use of sterile techniques including the wearing of sterile gloves during catheter assembly and sample collection and capping the syringe with a sterile cap are all important in this. In addition to maintaining sterility and preventing cross-contamination, particular challenges associated with processing and isolation of DNA from small intestinal samples include the viscosity and non-homogeneity of the samples, as well as small sample volumes coupled with lower microbial content. Recently, techniques and adaptations have been described aimed at optimizing DNA sequencing from ‘low-biomass’ samples.

High throughput DNA sequencing of biological samples such as body fluids (small bowel aspirates) for microbial population analysis often requires a certain amount of DNA, enough to build DNA libraries. In particular, small bowel aspirate samples have lower microbial density (low biomass) in normal conditions which make it very difficult to get enough DNA from microbial population. Further, small bowel aspirate itself is viscous, mainly because of the presence of glycoproteins (mucins). Thus, microbes can be trapped in the mucous part of the aspirate, decreasing the chances to get the entire population of microbes properly, during the DNA extraction first step procedure (to get the pellet of the samples).

Similarly, many biological samples are stored in conservation reagent Allprotect® reagent, which is also a viscous reagent, and can hinder the DNA extraction process.

As such there is a need in the art for methods of processing biological samples, and viscous biological samples in particular, and biological samples stored in conservation reagent such as Allprotect® regent to enable sufficient extraction of DNA.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of increasing the number of microorganisms extracted from a biological sample, comprising: adding a reducing agent to the biological sample to produce a mixture; and centrifuging the mixture to produce a supernatant and a pellet, wherein the number of microorganisms extracted is greater than the number of microorganisms extracted without the addition of a reducing agent.

In various embodiments, the biological sample can be a viscous biological sample. In various embodiments, the biological sample can be previously stored with a preservation and/or stabilization reagent.

In various embodiments, the reducing agent can be selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa, and combinations thereof. In particular embodiments, the reducing agent can be dithiothreitol (DTT). In particular embodiments, the reducing agent can be N-acetylcysteine (NAC).

In various embodiments, the method can further comprise conserving the pellet in a preservation and/or stabilization reagent. In various embodiments, the preservation and/or stabilization reagent can be Allprotect® reagent.

In various embodiments, the viscosity of the biological sample can be reduced.

In various embodiments, the biological sample can be selected from the group consisting of large or small intestinal fluids or aspirate, stomach fluids or aspirate, mucus, mucous membrane, stool, whole blood, plasma, serum, cerebral spinal fluid (CSF), urine, sweat, saliva, tears, pulmonary secretions, breast aspirate, prostate fluid, seminal fluid, cervical scraping, vaginal secretions, amniotic fluid, semen, synovial fluid, intraocular fluid, moisture in breath, sputum, liquid sample from inflamed tissue, and combinations thereof. In various embodiments, the biological sample can be selected from the group consisting of intestinal biopsies, small or large intestinal fluids or aspirates, and combinations thereof. In various embodiments, the small intestinal fluids or aspirates can be duodenal fluids or aspirate. In various embodiments, the biological sample can be selected from the group consisting of urine, saliva, sputum, liquid samples from inflamed tissue, mucous membranes, amniotic fluid, vaginal secretions, semen, synovial fluid, stool.

In various embodiments, the method can further comprise performing DNA extraction, DNA library preparation, DNA quantification, and/or DNA sequencing on the pellet.

Various embodiments of the present invention provide for a method, comprising: adding a reducing agent to a biological sample to produce a mixture; and centrifuging the mixture to produce a supernatant and a pellet.

In various embodiments, the reducing agent can be selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa, and combinations thereof.

In particular embodiments, the reducing agent can be dithiothreitol (DTT). In particular embodiments, the reducing agent can be N-acetylcysteine (NAC).

In various embodiments, the method can further comprise conserving the pellet in a preservation and/or stabilization reagent. In various embodiments, the preservation and/or stabilization reagent can be Allprotect® reagent.

In various embodiments, the viscosity of the biological sample can be reduced.

In various embodiments, the biological sample can be selected from the group consisting of large or small intestinal fluids or aspirate, stomach fluids or aspirate, mucus, mucous membrane, stool, whole blood, plasma, serum, cerebral spinal fluid (CSF), urine, sweat, saliva, tears, pulmonary secretions, breast aspirate, prostate fluid, seminal fluid, cervical scraping, vaginal secretions, amniotic fluid, semen, synovial fluid, intraocular fluid, moisture in breath, sputum, liquid sample from inflamed tissue, and combinations thereof. In various embodiments, the biological sample can be selected from the group consisting of intestinal biopsies, small or large intestinal fluids or aspirates, and combinations thereof. In various embodiments, the small intestinal fluids or aspirates can be duodenal fluids or aspirate. In various embodiments, the biological sample can be selected from the group consisting of urine, saliva, sputum, liquid samples from inflamed tissue, mucous membranes, amniotic fluid, vaginal secretions, semen, synovial fluid, stool.

In various embodiments, the method can further comprise performing DNA extraction, DNA library preparation, DNA quantification, and/or DNA sequencing on the pellet.

Various embodiments of the present invention provide for a method comprising: adding a reducing agent to a biological sample that was stored with a preservation and/or stabilization reagent to create a mixture; and centrifuging the mixture to produce a supernatant and a pellet.

In various embodiments, the reducing agent can be selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa, and combinations thereof. In particular embodiments, the reducing agent can be dithiothreitol (DTT). In particular embodiments, the reducing agent can be N-acetylcysteine (NAC).

In various embodiments, the method can further comprise performing DNA extraction, DNA library preparation and/or DNA sequencing on the pellet.

In various embodiments, the preservation and/or stabilization reagent can be Allprotect® reagent. In various embodiments, the viscosity of the biological sample and preservation and/or stabilization reagent mixture can be reduced.

In various embodiments, the biological sample can be selected from the group consisting of large or small intestinal fluids or aspirate, stomach fluids or aspirate, mucus, mucous membrane, stool, whole blood, plasma, serum, cerebral spinal fluid (C SF), urine, sweat, saliva, tears, pulmonary secretions, breast aspirate, prostate fluid, seminal fluid, cervical scraping, vaginal secretions, amniotic fluid, semen, synovial fluid, intraocular fluid, moisture in breath, sputum, liquid sample from inflamed tissue, and combinations thereof. In various embodiments, the biological sample can be selected from the group consisting of intestinal biopsies, small or large intestinal fluids or aspirates, and combinations thereof. In various embodiments, the small intestinal fluids or aspirates can be duodenal fluids or aspirate. In various embodiments, the biological sample can be selected from the group consisting of urine, saliva, sputum, liquid samples from inflamed tissue, mucous membranes, amniotic fluid, vaginal secretions, semen, synovial fluid, stool.

Various embodiments of the present invention provide for a kit, comprising: a quantity of a reducing agent; and instructions for using the quantity of the reducing agent to reduce the viscosity of a liquid sample.

In various embodiments, the kit can further comprise a preservation and/or stabilization reagent.

In various embodiments, the reducing agent can be selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa, and combinations thereof. In particular embodiments, the reducing agent can be dithiothreitol (DTT). In particular embodiments, the reducing agent can be N-acetylcysteine (NAC).

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a workflow for pretreatment and microbial culture, including the number of subjects in each group.

FIG. 2 depicts a workflow for DNA extraction and 16S sequencing of DA samples, including the number of subjects in each group.

FIG. 3 depicts final quantification of 16S libraries from DA-U and DA-DTT samples after 35 PCR cycles. The Mann-Whitney test was used to compare groups.

FIG. 4 depicts sunburst representation of the overall distribution of the small intestinal microbiome as determined by 16S rRNA sequencing. Left: Relative microbial abundance detected in DA-U (no pretreatment). Right: Relative microbial abundance detected in DA-DTT (pretreatment with DTT).

FIG. 5 depicts rarefaction curves for DA-DTT and DA-U. Samples were rarefied to the least numbers of sequences obtained.

FIG. 6 depicts Simpson's index and Shannon entropy diversity of DA-DTT (DTT pretreated DA) (n=43) and DA-U (non-pretreated DA) (n=112).

FIG. 7 depicts beta diversity of DA-U and DA-DTT based on the weighted UniFrac metric. Principal Coordinates Analysis plot of binary and abundance-weighted UniFrac distances between DA-DTT (unshaded circles, n=43) and DA-U (shaded circles, n=112).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“DNA” is meant to refer to a polymeric form of deoxyribonucleotides (i.e., adenine, guanine, thymine and cytosine) in double-stranded or single-stranded form, either relaxed or supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the non-transcribed strand of DNA (i.e., the strand having the sequence homologous to the mRNA). The term captures molecules that include the four bases adenine, guanine, thymine and cytosine, as well as molecules that include base analogues which are known in the art.

“Isolated” DNA, RNA, peptides, polypeptides, or proteins include DNA, RNA, peptides polypeptides or proteins that are isolated or purified relative to other DNA, RNA, peptides, polypeptides, or proteins in the source material.

The term “biological sample” as used herein denotes a sample taken or isolated from a biological organism. Further examples of biological samples include but are not limited to intestinal fluids or aspirate, and stomach fluids or aspirate, duodenal fluids or aspirate, mucus (from e.g., intestine, nose, lung, eye, urogenital systems), mucous membrane, stool, whole blood, plasma, serum, cerebral spinal fluid (CSF), urine, sweat, saliva, tears, pulmonary secretions, breast aspirate, prostate fluid, seminal fluid, cervical scraping, vaginal secretions, amniotic fluid, semen, synovial fluid, intraocular fluid, moisture in breath, sputum, and liquid sample from inflamed tissue. In particular embodiments of the method, the biological sample may be stool, intestinal fluid or aspirate or stomach fluid or aspirate, or duodenal fluids or aspirate. In various embodiments, the biological sample may be duodenal fluid or aspirate. In various embodiments, the biological sample may be intestinal fluid or aspirate. In various embodiments, the biological sample may be stomach fluid or aspirate. The term also includes a mixture of the above-mentioned samples.

Described herein, we developed and validated a technique to optimize microbial processing and DNA isolation and sequencing from viscous biological samples (e.g., viscous liquids), particularly, for duodenal aspirates.

The characterization of the microbiomes living in association with the human body is one of the actively developing field in research since the creation of the Human Microbiome Project in 2007. A decade ago very little was known about the microorganism composition in different human body sites and it relation with the human body physiology and pathologic processes, but with the launch of high throughput DNA sequencing technologies, and improvements in DNA preparation and computational methods, the human microbiomes have been extensively studied and previous works have been already demonstrated differences in the composition of host-associated microbial communities in healthy and disease states.

The quality of the microbiome data is associated in some way to each step from study design to analysis. The sample collection and processing prior sequencing are two critical steps in microbiome studies and the choose of proper techniques and kits can impact results. The nature of microbiome samples collected from different parts of the body changes, and some of them require special handling and optimized protocols for DNA extraction and sequencing library preparation, considering factors such as sample viscosity, quantity, cell host contamination, low microbial biomass, etc. The upper portions of human small intestine are not populated as much as the large intestine and samples collected from duodenum have different viscosity.

Described herein, we developed and validated a methodological approach based on the use of the reducing agent dithiothreitol (DTT) that resolves issues related to low microbial biomass from luminal duodenal aspirates (DA). The use of DTT clearly increases the number of bacteria detected on culture plates, and also increases DNA yields and the concentration of V3/V4 libraries for sequencing, which in turn results in important differences in the microbial populations detected in DA. Other reducing agents that can be used include but are not limited to 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, and Urea. Additional reducing agents that can be used include but are not limited to N-acetylcysteine (NAC), acetylcysteine, ambroxol, bromhexine, carbocisteine, erdosteine, mecysteine, dornase alfa and combinations thereof. In various embodiments, the reducing agent is dithiothreitol (DTT). In various embodiments, the reducing agent is N-acetylcysteine (NAC).

Given the central role of the small intestine in the processes of digestion and nutrient absorption, accurate characterization of the human small intestinal microbiome is an important future consideration. The small intestine is not as heavily colonized as the large intestine, ranging under healthy conditions from 10³-10⁴ bacteria per mL of intestinal content in the duodenum and jejunum to 10⁸ bacteria per mL in the ileum, and 10¹¹ bacteria per gram of wet stool in the colon. In addition to having low bacterial biomass, duodenal luminal contents are viscous due to the mucus layer present in the small intestine, and require special handling during sample collection and processing prior to culture and DNA extraction, in order to increase the likelihood of the assessment of all microbial communities, including those associated with the mucus layer.

In the assessment of the microbiome, it is essential to accurately and completely assess the microbial components in a sample. Although standards have been set for the assessment of the stool microbiome, these standards have not been assessed for small intestinal fluid assessment. Mucous in general is a viscous fluid that can trap bacteria in its matrix and previous studies performed with sputum samples have shown whether treating this viscosity has an impact on the microbial assessment. However, until now, no studies have investigated the impact on microbial assessment and DNA recovery in aspirates collected from small bowel.

Various embodiments herein describe agents that safely and effectively improve microbial assessment and DNA yield in viscous samples, and one such agent is DTT, which can reduce the disulfide bonds between mucin subunits.

We established a methodology to improve microbial DNA recovery from small bowel aspirates, which includes different sample processing steps when compared to conventionally published methods for extracting DNA for microbiome assessment of gut materials such as stool. The concentrations of DNAs extracted from DA ranged from very low levels to up to 70 ng/ml when samples were pretreated with DTT, more than 3-fold higher than those from samples which were not pretreated with the reducing agent. Initial DNA concentrations exhibited a higher positive correlation with those of the final V3/V4 libraries for DTT-pretreated DA compared to non-pretreated DA, which may indicate a specific increase in the isolation of bacterial DNAs. The use of a fixed initial DNA concentration during the preparation of sequencing libraries from DA should be carefully analyzed, and the addition of DTT during sample processing and for removal of the ALL PROTECT reagent is highly recommended as this increases the initial yield of microbial DNA prior sequencing library preparation.

In addition to increases in DNA yields and library concentrations, DA processed with DTT prior to microbial culture exhibited higher numbers of bacterial colonies on blood agar plates incubated under anaerobic conditions. The relative abundance of specific obligate and facultative anaerobes detected in DA-DTT was increased compared to DA-U samples, which may reflect the increased detection of microbes associated with the mucus layer. For example, the relative abundance of the genus Enterococcus (phylum Firmicutes) detected in DA-DTT was significantly increased. This genus comprises over 50 Gram-positive facultative anaerobic lactic acid cocci species isolated from numerous environments, including the human GI tract. Enterococcus species constitute up to 1% of the gut microbiota, and most species can grow on blood agar plates under anaerobic conditions. The relative abundance of the genus Clostridium (phylum Firmicutes), which is comprised of obligate anaerobes and some aerotolerant species, detected in DA-DTT samples was also increased.

The relative abundance of the genus Bacteroides, comprised of Gram-negative obligately anaerobic bacilli, detected in DA-DTT was also significantly increased compared to DA-U samples. Species from this genus can grow on blood agar and are well-adapted to the gastrointestinal tracts of mammals, including humans. The human large intestine is densely colonized with species from the genus Bacteroides (phylum Bacteroidetes), many of which perform essential metabolic functions for the host, including the metabolism of proteins and complex sugars. In contrast, the small intestine is not as heavily colonized by members of the phylum Bacteroidetes, which comprised less than 4% of the total microbes detected in DA-U. With the addition of the reducing agent DTT, which breaks the disulfide bonds linking mucin subunits in mucus prior to microbial culture and DNA extraction, the relative abundance of Bacteroidetes detected in DA increased significantly from 4% to 7%, indicating a possible role for species from this phylum in mucus metabolism. Mucus is a dynamic matrix, consisting of mucin glycoproteins secreted by intestinal goblet cells, which lubricates the transit of intestinal contents, amongst other functions. Mucus glycoproteins can be used as a carbon source by many asaccharolytic microorganisms, and the low oxygen levels at atmospheric pressures allow the colonization and growth of anaerobes in mucus.

The phylum Proteobacteria also includes aerotolerant asaccharolytic microorganisms that require proteinaceous substrates as carbon and energy sources, such as Campylobacter, as well as facultative anaerobes from the family Enterobacteriaceae included in the “Mucosally Associated Consortium” in the colon, described by Albenberg et al.

We show herein that pretreatment of DA with DTT increases the detected relative abundance of many Enterobacteriaceae members, including the clinically important genera Klebsiella, Providencia and Salmonella as well as unknown members. Providencia and Salmonella include motile species that can adhere to mucus and epithelial cells and actively invade the host epithelium. The relative abundance of the genus Pseudomonas, detected in DA-DTT was also increased compared to non-pretreated DA. Members of this genus, including the most studied species P. aeruginosa, are also motile and can be part of the normal human microflora, but are also important clinically as they are known to cause hospital-acquired infections such as pneumonia and urinary tract infections.

The changes in several microbial taxa in DA samples after the addition of DTT did not affect the overall microbial diversity. These findings further show that the addition of the reducing agent DTT improves microbial assessment and DNA recovery without causing a dramatic change in the microbial balance in the aspirate samples.

Further described herein we validate the methodology to optimize yield for culture and for DNA extraction for analysis of the small bowel microbiome. Culture totals, microbial DNA and microbiome analysis demonstrate marked differences with this new technique. This suggests that conventional techniques for DNA isolation provide an incomplete picture of the microbial environment in the small bowel. Thus, this technique is ideal for small bowel microbiome assessment.

Various embodiments of the present invention are based, at least in part, on these findings.

Various embodiments of the present invention provide for a method of reducing the viscosity of a biological sample. Reducing the viscosity of biological samples enable higher quantities of DNA extraction from the biological sample. The method comprises: adding a reducing agent to a biological sample to produce a mixture; and centrifuging the mixture to produce a supernatant and a pellet. In various embodiments, the viscosity of the biological sample is reduced.

In various embodiments, the method further comprising conserving the pellet in a preservation and/or stabilization reagent.

In various embodiments, the method further comprising performing DNA extraction, DNA library preparation and/or DNA sequencing on the pellet.

In various embodiments, the reducing agent is dithiothreitol (DTT). In various embodiments, the reducing agent is selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa and combinations thereof. In particular embodiments, the reducing agent is NAC.

In various embodiments, the preservation and/or stabilization reagent is Allprotect® reagent.

In various embodiments, the biological sample is selected from the group consisting of large or small intestinal fluids or aspirate, stomach fluids or aspirate, mucus, mucous membrane, stool, whole blood, plasma, serum, cerebral spinal fluid (CSF), urine, sweat, saliva, tears, pulmonary secretions, breast aspirate, prostate fluid, seminal fluid, cervical scraping, vaginal secretions, amniotic fluid, semen, synovial fluid, intraocular fluid, moisture in breath, sputum, liquid sample from inflamed tissue, and combinations thereof. In various embodiments, the biological sample is selected from the group consisting of intestinal biopsies, small or large intestinal fluids or aspirates, and combinations thereof. In various embodiments, the biological sample is selected from the group consisting of intestinal biopsies, small bowel aspirates and combinations thereof. In various embodiments, the small intestinal fluids or aspirates is duodenal fluids or aspirate. In various embodiments, the biological sample is selected from the group consisting of urine, saliva, sputum, liquid samples from inflamed tissue, mucous membranes, amniotic fluid, vaginal secretions, semen, synovial fluid, stool.

Various embodiments of the present invention provide for a method of reducing the viscosity of a mixture of a biological sample stored in a preservation and/or stabilization reagent. The method comprises adding a reducing agent to a biological sample that was stored with a preservation and/or stabilization reagent to create a mixture; and centrifuging the mixture to produce a supernatant and a pellet. In various embodiments, the viscosity of the biological sample and preservation and/or stabilization reagent mixture is reduced.

Various embodiments of the present invention provide for a method of increasing the number of microorganisms extracted a biological sample, comprising: adding a reducing agent to the biological sample to produce a mixture; and centrifuging the mixture to produce a supernatant and a pellet, wherein the number of microorganisms extracted is greater than the number of microorganisms extracted without the addition of a reducing agent.

In various embodiments, the method further comprises performing DNA extraction, DNA library preparation and/or DNA sequencing on the pellet.

In various embodiments, the reducing agent is dithiothreitol (DTT). In various embodiments, the reducing agent is dithiothreitol (DTT). In various embodiments, the reducing agent is selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa and combinations thereof. In particular embodiments, the reducing agent is NAC.

In various embodiments, the preservation and/or stabilization reagent is Allprotect® reagent.

In various embodiments, the biological sample is selected from the group consisting of large or small intestinal fluids or aspirate, stomach fluids or aspirate, mucus, mucous membrane, stool, whole blood, plasma, serum, cerebral spinal fluid (CSF), urine, sweat, saliva, tears, pulmonary secretions, breast aspirate, prostate fluid, seminal fluid, cervical scraping, vaginal secretions, amniotic fluid, semen, synovial fluid, intraocular fluid, moisture in breath, sputum, liquid sample from inflamed tissue, and combinations thereof. In various embodiments, the biological sample is selected from the group consisting of intestinal biopsies, small or large intestinal fluids or aspirates, and combinations thereof. In various embodiments, the biological sample is selected from the group consisting of intestinal biopsies, small bowel aspirates and combinations thereof. In various embodiments, the small intestinal fluids or aspirates is duodenal fluids or aspirate. In various embodiments, the biological sample is selected from the group consisting of urine, saliva, sputum, liquid samples from inflamed tissue, mucous membranes, amniotic fluid, vaginal secretions, semen, synovial fluid, stool.

Various embodiments of the present invention provide for a kit for reducing the viscosity of a biological sample, or for reducing the viscosity of a mixture of a biological sample stored in a preservation and/or stabilization reagent. The kit comprises a quantity of a reducing agent; and instructions for using the quantity of the reducing agent to reduce the viscosity of a biological sample, or for reducing the viscosity of a mixture of a biological sample stored in a preservation and/or stabilization reagent. In various embodiments, the reducing agent is dithiothreitol (DTT).

In various embodiments, the kit further comprises a preservation and/or stabilization reagent. In various embodiments, the reducing agent is dithiothreitol (DTT). In various embodiments, the reducing agent is dithiothreitol (DTT). In various embodiments, the reducing agent is selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa and combinations thereof. In particular embodiments, the reducing agent is NAC.

The kit is an assemblage of materials or components, including at least one of the inventive compositions. Thus, in some embodiments the kit contains a composition including the reducing agent, or the reducing agent and preservation and/or stabilization reagent, as described above.

The exact nature of the components configured in the inventive kit depends on its intended purpose. For example, some embodiments are configured for the purpose of reducing the viscosity of a biological sample; and other embodiments are configured for the purpose of reducing the viscosity of a mixture of a biological sample stored in a preservation and/or stabilization reagent. In one embodiment, the kit is configured particularly for the purpose of processing mammalian biological samples. In another embodiment, the kit is configured particularly for the purpose of processing human biological samples. In another embodiment, the kit is configured particularly for the purpose of processing intestinal biopsies or small intestinal aspirates. In further embodiments, the kit is configured for veterinary applications, processing samples from subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to reduce the viscosity of the sample. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as reducing agents and the like. The packaging material is constructed by well-known methods, preferably to provide a sterile, contaminant-free environment. The packaging materials employed in the kit are those customarily utilized in biological tissue and sample processing. As used herein, the term “package” refers to a suitable solid matrix or material such as glass, plastic, paper, foil, and the like, capable of holding the individual kit components. Thus, for example, a package can be a glass vial used to contain suitable quantities of a reducing agent. In another example, the package can include centrifuge tubes containing measured quantities of the reducing agent. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

We performed our DNA 16S sequencing of small bowel aspirate samples. The DNA was extracted using a well-known and established protocols without any changes. We also prepared DNA libraries with a well-known and established library preparation kit without any modification. We performed a quality analysis after the library preparation and the expected DNA fragment did not show up. Even so, we performed the 16S sequencing and it did not work. Based on that we decided to change the sample processing method before DNA sequencing. High throughput DNA sequencing of biological samples such as body fluids (small bowel aspirates) for microbial population analysis often requires a certain amount of DNA, enough to build DNA libraries. In particular, small bowel aspirate samples have lower microbial density (low biomass) in normal conditions which make it very difficult to get enough DNA from microbial population. Besides that, the small bowel aspirate itself is viscous, mainly because of the presence of glycoproteins (mucins). Because of that, microbes can be trapped in the mucous part of the aspirate, decreasing the chances to get the entire population of microbes properly, during the DNA extraction first step procedure (to get the pellet of the samples). Taking those two facts together (low biomass and viscosity), we decide to look for options to increase our chances to get enough DNA. We did not find any specific kit for that purpose so we had to develop a new strategy based on previous knowledge and publications.

We created a new strategy to process the aspirates samples with DTT and increase the changes to obtain enough DNA for sequencing.

We looked for solutions combining the reducing reagent and DNA extraction method for low biomass samples. We could not find a single protocol, so we combined the liquefaction step with DTT to reduce viscosity from pure aspirates and aspirates stored with ALL PROTECT and a DNA extraction bead-based kit for soil samples.

### Example 2

We first tested five samples. All steps, including samples processing, DNA extraction, DNA library construction, quality control analysis and high throughput sequencing took 20 days, with a commitment of 8 to 10 hours per day.

We obtained the first results of our combined methodology, described briefly below:

The small bowel aspirate samples were first treated with DTT to decrease viscosity. After centrifuged at maximum speed, the supernatant was removed and the pellet was conserved in ALL PROTECT reagent until DNA extraction procedure. The ALL PROTECT reagent itself has high viscosity so the DTT was added again to the samples conserved in ALL PROTECT. After centrifuge again at maximum speed and get the microbial pellet, the DNA was extracted using a well-known established protocol for soil samples. The libraries were constructed and the first quality analysis results were reviewed. All samples passed in the quality analysis step, showing the expected DNA fragment on the right position. The samples were finally sequenced the partial results were reviewed. We have already sequenced more than 50 aspirate samples as of August 2018.

### Example 3

### Study Subjects

Male and female subjects aged 18-85 undergoing esophagogastroduodenoscopy without colon preparation for standard of care purposes were prospectively recruited for this study. Potential participants were identified by study staff and their eligibility was verified by co-investigators or the PI. Although there were no exclusion criteria, small bowel biopsies were not collected from subjects with bleeding disorders or advanced cirrhosis of the liver with coagulopathy and intestinal varices where the international normalized ratio (INR) was greater than 1.5, in order to minimize the risk of bleeding from the biopsy site. The study protocol was approved by the Institutional Review Board at Cedars-Sinai Medical Center, and all subjects provided informed written consent prior to participating in the study. Samples from subjects taking antibiotics were not included in the present study.

### Questionnaires

Prior to endoscopy, all subjects completed a study questionnaire which documented their demographics and medical and family history, including GI disease, medication use, use of alcohol and recreational drugs, travel history, and dietary habits and changes. Medical information provided by participants was verified using medical records. All patient data were de-identified prior to analysis.

### Small Intestinal Sample Procurement—Aspirates

During the esophagogastroduodenoscopy procedure, samples of luminal fluid (˜2 ml) were obtained from the duodenum using a custom sterile aspiration catheter (Hobbs Medical, Inc.). The custom catheter consisted of a newly designed double lumen sterile catheter, with the inner lumen maintaining sterility during insertion by applying sterile bone wax into the open tip of the external catheter. During endoscopy, the endoscopist is instructed to immediately enter the duodenum and insert the aspiration catheter. The inner catheter then dislodges the bone wax, exposing the sterile inner catheter. This inner catheter is used to aspirate duodenal fluid through lasered side holes to acquire a volume of 2 mL. These precautions eliminated the risk of oral and gastric contamination.

### Aspirate Microbial Culture and Processing

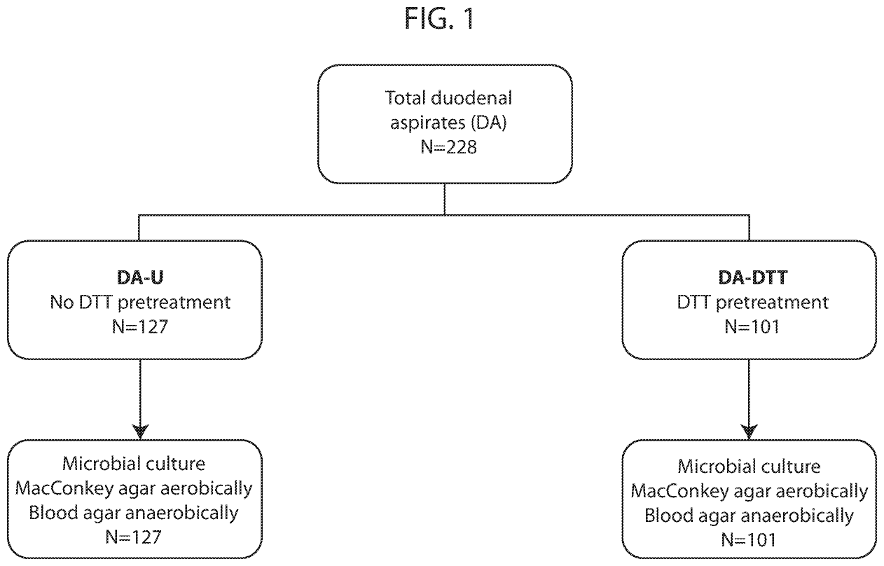

Immediately after aspiration, samples from all duodenal aspirates were cultured on MacConkey agar (Becton Dickinson, Franklin Lakes, N.J., EUA) and blood agar (Becton Dickinson) for determination of the number of colony-forming units (CFU) per mL of aspirate. To assess the effect of viscosity on culture, a subset of aspirates was not pretreated and simply cultured (the DA-U group for “untreated”) and another subset were first pretreated with the reducing agent Dithiothreitol (DTT) (Sputolysin® Reagent, Cat. 560000-1SET, EMD Millipore Corp, Billerica, Mass., USA) (the DA-DTT group) (see FIG. 1).

For DA-DTT samples, 1×DTT (6.5mM dithiothreitol in 100 mM phosphate buffer, pH 7.0) was added to an aliquot of the DA in a 1:1 ratio and the resulting mixture was vortexed until the sample was liquified (typically 30 seconds). 100 μL of the liquified mixture was serially diluted with 900 μL sterile 1×PBS and samples of the 1:10 and 1:100 dilutions were plated in duplicate on MacConkey agar under aerobic conditions for the quantitation of Gram-negative bacilli, and on blood agar under anaerobic conditions for the quantitation of total anaerobes. For DA-U samples, 100 μL of DA was diluted directly with 900 μL sterile 1×PBS and samples of the 1:10 and 1:100 dilutions were plated as described above. All plates were incubated at 37° C. for 16-18 hours, after which colonies were electronically counted using the Scan 500 (Interscience, Paris, France). As a negative control, 100 μL of 1×DTT was also cultured aerobically on MacConkey agar and anaerobically on blood agar. All dilution factors were taken into account for final determination of microbial burden.

After aliquots for microbial culture were taken, remaining DA-U and DA-DTT samples were centrifuged at high speed (17136×g) for 10 minutes and the supernatant was carefully removed and stored at −80° C. for future metabolomic analyses. 500 μL of sterile ALL PROTECT reagent (Qiagen, Hilden, Germany) was added to each pellet for stabilization of DNA, RNA and proteins, and the pellets were stored at −80° C. prior to DNA isolation and analysis of the DA microbiome.

### DNA Extraction and Quantification—Duodenal Aspirates

DA-U and DA-DTT samples were thawed on ice and 1×DTT was added in a 1:1 ratio, after which the samples were vortexed until the ALL PROTECT reagent was fully liquefied (around 30 seconds). DNA extraction was then performed for both groups using the MagAttract PowerSoil DNA KF Kit (Qiagen, cat. No. 27000-4-KF) with some modifications. DNA extraction was also performed on negative control samples (1×DTT) as a control.

The lysis step was carried out by adding garnet beads (Qiagen, cat. No. 13123-50) and 746 μL PowerBead Solution to each pellet-containing tube, followed by 4 μL RNase A and 60 μL SL Solution (Lysis buffer) in this specific order. Tubes were sealed with parafilm, vortexed horizontally for 15 minutes, and then centrifuged for 6 minutes at 4500×g. The supernatants were transferred to new tubes containing 450 μL IR Solution, vortexed for 3 seconds, incubated at 4° C. for 10 minutes, and then centrifuged for 6 minutes at 4500× g. The supernatants were transferred to new tubes and centrifuged for a further 6 minutes at 4500×g. 450 μL of the resulting supernatants were added to deep 96-well KingFisher plates containing magnetic beads and DNA extraction was performed using the KingFisher Duo (Thermo Fisher Scientific, Waltham, Mass., USA). The final DNA volume was 100 μL. DNAs were then quantified using Qubit ds DNA BR Assay kits (Invitrogen by Thermo Fisher Scientific, Waltham, Mass., USA) on a Qubit 4 Fluorometer (Invitrogen).

### Library Preparation and 16S rRNA Sequencing

16S library preparation for DNAs from all groups was performed according to the Illumina (Illumina, San Diego, Calif., USA) protocol support.illumina.com/documents/documentation/chemistry documentation/16s/16s-metagenomic-library-prep-guide-15044223-b.pdf, with some modifications. The V3 and V4 regions were amplified using the gene-specific primers S-D-Bact-0341-b-S-17 and S-D-Bact-0785-a-A-21 published and validated by Klindworth et al. The primers were modified in accordance with the protocol by adding the Illumina sequencing adapters to each one.

The full-length primer sequences used were:

16S Amplicon PCR Forward Primer:
(SEQ ID NO: 1)
5′ TCGTCGGCAGCGTCAGATGTGTA
TAAGAGACAGCCTACGGGNGGCWGCAG;
16S Amplicon PCR Reverse Primer:
(SEQ ID NO: 2)
5′ GTCTCGTGGGCTCGGAGATGTGTAT
AAGAGACAGGACTACHVGGGTATCTAATCC

The 16S library preparation protocol was modified as follows: 5 μl of DNA was added to a Master Mix (0.5 μl of 10 μM 16S Amplicon PCR Forward primer, 0.5 μl of 10 μM 16S Amplicon PCR Reverse primer, 12.5 μl 2×KAPA HiFi HotStart ReadyMix and 6.5 μl of molecular grade PCR H₂O) and the PCR was performed as follows:
1. Initial denaturation step at 95° C. for 3 minutes
2. 27 cycles of: 95° C. for 30 seconds, 55° C. for 30 seconds and 72° C. for 30 seconds
3. 72° C. for 5 minutes
4. Hold at 4° C.

An optimized Clean-Up step was performed with Agencourt AMPure XP beads using the modifications proposed by Quail et al. After adding the beads to each Amplicon PCR 96-well plate on the magnetic stand, samples were incubated for five minutes followed by two wash steps with 80% ethanol. The beads were air dried for five minutes. After removing the plate from magnetic stand, beads were incubated with EB Buffer (Qiagen) for five minutes to elute the DNA. The plate was placed back on the magnetic stand and after 2-3 minutes the supernatant was transferred to an empty clean well, preventing the transfer of the beads with the supernatant.

Five μl of the final Amplicon PCR product was used for the Index PCR, which was performed using the Nextera XT Index kit and 2×KAPA HiFi HotStart ReadyMix, following the Illumina protocol. After a second modified Clean-Up step, the final product was quantified using Qubit ds DNA BR Assay kits and Qubit 1×dsDNA HS Assay kits on a Qubit 4 Fluorometer and analyzed using Agilent DNA 1000 chips (Agilent Technologies, Santa Clara, Calif.) and Agilent HS DNA chips (Agilent) on an Agilent 2100 Bioanalyzer System.

### DTT-Pretreated and Non Pretreated DA DNA Samples

16S library preparation for DNAs from DTT-pretreated and non-pretreated DA samples was performed according to the protocol described above for stool and naïve DNA samples, with some additional modifications. The V3 and V4 regions were amplified using the same set of gene-specific primers.

The 16S library preparation protocol was modified as follows: 5 μl of DNA was added to a Master Mix (0.5 μl of 10 μM 16S Amplicon PCR Forward primer, 0.5 μl of 10 μM 16S Amplicon PCR Reverse primer, 12.5 μl 2×KAPA HiFi HotStart ReadyMix and 6.5 μl of molecular grade PCR H₂O) and the PCR was performed as follows:
1. Initial denaturation step at 95° C. for 3 minutes
2. 27 cycles of: 95° C. for 30 seconds, 55° C. for 30 seconds and 72° C. for 30 seconds
3. 72° C. for 5 minutes
4. Hold at 4° C.

An optimized Clean-Up step was performed with Agencourt AMPure XP beads using the modifications proposed by Quail et al. After adding the beads to each Amplicon PCR 96-well plate on the magnetic stand, samples were incubated for five min followed by two wash steps with 80% ethanol. The beads were air dried for five min. After removing the plate from magnetic stand, beads were incubated with EB Buffer (Qiagen) for five minutes to elute the DNA. The plate was placed back on the magnetic stand and after 2-3 minutes the supernatant was transferred to an empty clean well, preventing the transfer of the beads with the supernatant.

Five μl of the final Amplicon PCR product was used for the Index PCR, which was performed using the Nextera XT Index kit and 2×KAPA HiFi HotStart ReadyMix, following the Illumina protocol for 8 cycles. After a second modified Clean-Up step, the final product was quantified using Qubit ds DNA BR Assay kits and Qubit 1×dsDNA HS Assay kits on a Qubit 4 Fluorometer and analyzed using Agilent DNA 1000 chips (Agilent Technologies, Santa Clara, Calif.) and Agilent HS DNA chips (Agilent) on an Agilent 2100 Bioanalyzer System.

### 16S Metagenomic Sequencing and Analysis

The V3 and V4 libraries prepared using DNAs from DA-DTT and DA-U groups were sequenced using a MiSeq Reagent Kit v3 (600-cycles) on a MiSeq System (Illumina, San Diego, Calif.). 2×301 cycles paired-end sequencing was performed according to manufacturer's protocol and 5% Phix (Illumina) was added to each library pool.

Operational Taxonomic Unit (OTU) clustering and taxonomic analyses were performed using CLC Genomics Workbench v. 10.1.1 and CLC Microbial Genomics Module v. 2.5 (Qiagen). Sequences were first trimmed to remove 13 bases at the 5′ terminal position and merged considering the alignment scores as follows: mismatch cost of 2, gap cost of 2, zero maximum unaligned end mismatches and minimum score of 30. After merging, sequences were clustered into OTUs at 97% sequence similarity level using the Amplicon-Based OTU clustering tool. The creation of new OTUs was allowed considering 97% taxonomic similarity. The most abundant sequences were selected as representative of each cluster, and then assigned to a taxonomy level using CLC Microbial Genomics default values and the Greengenes Database 2013 release. Low depth samples (less than 9,000 sequences per sample) were removed from the analysis. Alpha diversity indexes (Chaol, Simpson and Shannon) were calculated using the Abundance Analysis tool. The weighted Unifrac metric was used to calculate inter-sample diversity (beta diversity).

### Statistical Analysis

Multiple comparisons and statistical analyses were performed using CLC Genomics Workbench v. 10.1.1 and CLC Microbial Genomics Module v. 2.5 (Qiagen). A Negative Binomial Generalized Linear Model (GLM) model was used to obtain maximum likelihood estimates for an OTU's log-fold change between two conditions, and the Wald test was used to determine significance, as part of the CLC package available at www.qiagenbioinformatics.com/products/clc-genomics-workbench/. False Discovery Rate (FDR) was performed to correct P-values. Fold changes are calculated from the GLM, which corrects for differences in library size between the samples and the effects of confounding factors. Again, these calculations were performed using the CLC package. It is therefore not possible to derive these fold changes from the original counts by simple algebraic calculations. Two-tailed Spearman r correlations, Mann-Whitney tests and graph construction were performed using GraphPad Prism 7.02 (GraphPad Software, La Jolla, Calif., USA). For statistical analysis purposes only, no growth on blood agar and MacConkey agar (CFU/ml=0) was assigned as 1 CFU/ml.

### PICRUSt Analysis for Predicted Metabolic Functions

The predicted metabolic functions of the microbial communities were analyzed using the Phylogenetic Investigation of Communities by Reconstruction of Unobserved States package (PICRUSt v1.1.3). The analysis was conducted with OTUs closed-referenced picked against the Greengenes (v13.5) database at a 97% identity. The prediction of metabolic functions was performed using the Kyoto Encyclopedia of Genes and Genome (KEGG) Orthology (KO) Database7. The PICRUSt accuracy was estimated using weighted Nearest Sequenced Taxon Index (weighted NSTI) values, where low NSTI values imply high accuracy of the predicted KEGG functional groups. Statistical comparisons of predicted metabolic functions in DA-DTT versus DA-U were carried out using the Mann-Whitney test in IBM® SPSS® version 24.

### Samples and Treatment

A total of 228 subjects had DA were collected and analyzed as shown in FIG. 1. Of these, 127 DA were not pretreated with DTT prior to microbial culture (DA-U, the untreated group), and 101 were pretreated with DTT prior to microbial culture (the DA-DTT group).

### DTT Effect on Microbial Cultures

No growth was observed in MacConkey agar plated with 1×DTT only (negative control). The CFU on MacConkey agar obtained from DA-U subjects ranged from 1 to 240×10³ CFU/mL (Mean=12.6×10³ CFU/mL, Median=1 CFU/mL, 25th percentile=1 CFU/mL and 75th percentile=1 CFU/mL). In the DA-DTT group, the CFU on MacConkey agar ranged from 1 to 3323×10³ CFU/mL (Mean=55.43×10³ CFU/mL, Med=1 CFU/mL, 25th percentile=1 CFU/mL and 75th percentile=1.1×10³ CFU/mL). DA-DTT exhibited 4.87-fold greater bacterial colonies on MacConkey agar when compared to DA-U, but the p-value did not reach statistical significance (P=0.16).

No growth was observed on blood agar cultured with 1×DTT only (negative control). The CFU on blood agar obtained from DA-U subjects ranged from 1 to 260×10³ CFU/mL (Mean=13.26×10³ CFU/mL, Median=1 CFU/mL, 25th percentile=1 CFU/mL and 75th percentile=1.1×10³ CFU/mL). On blood agar, DA-DTT exhibited 6.84-fold greater anaerobic bacterial colonies when compared to DA-U (P=0.0001). In the DA-DTT group, CFU on blood agar ranged from 1 to 2070×10³ CFU/ml (Mean=90.76×10³ CFU/mL, Med=1 CFU/mL, 25th percentile=1 CFU/mL and 75th percentile=95×10³ CFU/mL).

### DTT Effect on Microbial Cultures (Alternate Analysis)

No growth was observed in MacConkey agar plated with 1×DTT only (negative control). The CFU on MacConkey agar obtained from DA-U subjects ranged from 0 to 240×10³ CFU/mL (Mean=10.6×10³ CFU/mL, Median=0 CFU/mL, 25th percentile=0 CFU/mL and 75th percentile=0 CFU/mL). In the DA-DTT group, the CFU on MacConkey agar ranged from 0 to 1035×10³ CFU/mL (Mean=28.22×10³ CFU/mL, Med=0 CFU/mL, 25th percentile=0 CFU/mL and 75th percentile=2.5×10³ CFU/mL). For the purposes of statistical analysis only, no growth was designated as 1 bacterial CFU/mL of aspirate. DA-DTT exhibited 2.6-fold greater bacterial colonies on MacConkey agar when compared to DA-U, but the p-value did not reach statistical significance (P=0.14).

No growth was observed on blood agar cultured with 1×DTT only (negative control). The CFU on blood agar obtained from DA-U subjects ranged from 0 to 800×10³ CFU/mL (Mean=31.3×10³ CFU/mL, Median=0 CFU/mL, 25th percentile=0 CFU/mL and 75th percentile=20×10³ CFU/mL). On blood agar, DA-DTT exhibited 2.86-fold greater anaerobic bacterial colonies when compared to DA-U (P=0.0101). For the purposes of statistical analysis only, no growth was designated as 1 bacterial CFU/mL of aspirate. In the DA-DTT group, CFU on blood agar ranged from 0 to 2070×10³ CFU/ml (Mean=89.58×10³ CFU/mL, Med=6×10³ CFU/mL, 25th percentile=0 CFU/mL and 75th percentile=98.5×10³ CFU/mL).

### Immediate Post Aspiration DTT Improves DNA Extraction and 16S Metagenomic Library Preparation for DA

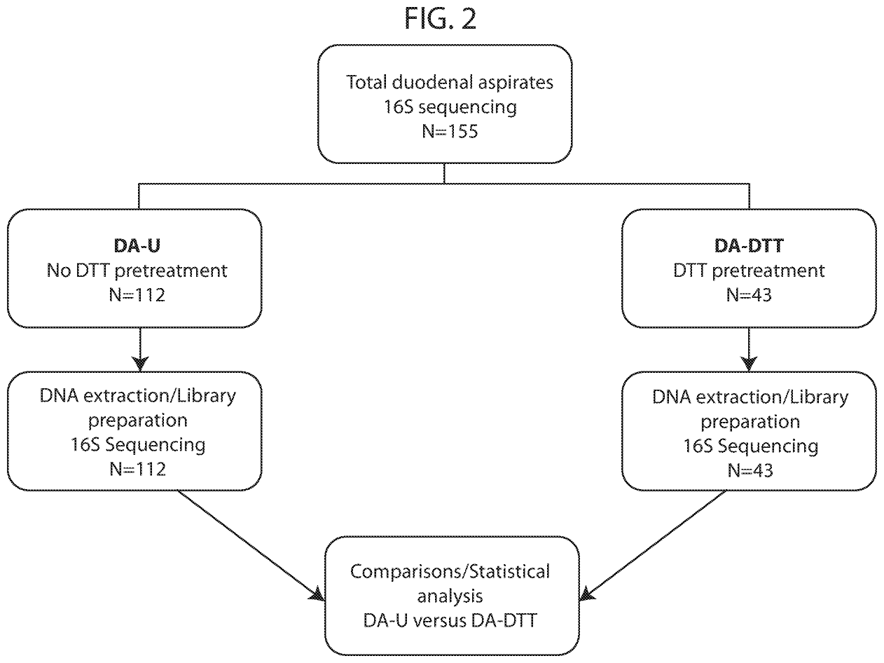

A total of 155 subject had their DA samples sequenced and analyzed as shown in FIG. 2. The concentrations of DNAs obtained from negative controls (DTT only) were undetectable. The concentrations of DNAs obtained from DA-U subjects ranged from undetectable levels (lower than 10 pg/μL (n=18) to 24.6 ng/μL (Med=0.0908 ng/μL, 25^{th} percentile=0.02365 ng/μL and 75^{th} percentile=0.6875 ng/μL). Treatment with DTT improved DNA isolation. In the DA-DTT group, DNA ranged from undetectable levels (n=3) to 68.8 ng/μL (Med=0.346 ng/μl, 25^{th} percentile=0.0906 ng/μL and 75^{th} percentile=1.91 ng/μL) and were 3.81-fold higher than those from DA-U (Mann Whitney P=0.0014).

The concentrations of the final 16S libraries amplified from negative controls were undetectable. The concentrations of the final 16S libraries amplified from DA-U samples (i.e., those for which DTT was added only for the removal of the ALL PROTECT reagent) ranged from 0.14 ng/μL to 136 ng/μL (median=21.8 ng/μl, 25^{th} percentile=5.1 ng/μL and 75^{th} percentile=69.8 ng/μL) and correlated with the initial DNA concentrations (Spearman r=0.316, P=0.001). Amplicons from DA-U libraries with low concentrations showed less intense fragments on the Bioanalyzer gel and some had no fragments of the expected size (˜630 bp).

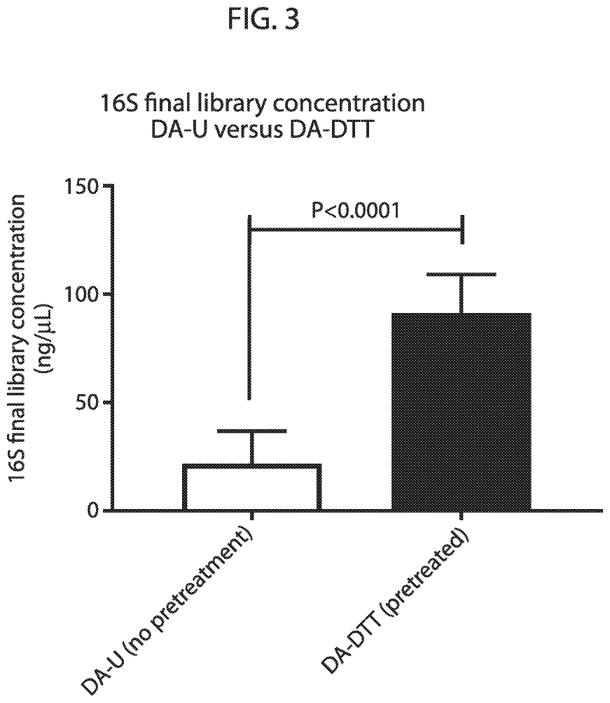

The concentrations of the final 16S libraries amplified from DA-DTT samples (i.e., those for which DTT was added both before microbial culture and for removal of the ALL PROTECT reagent) were 4.18× higher than those of libraries from DA-U samples (P<0.0001) (see FIG. 3). The library concentrations ranged from 1.69 ng/μL to 302 ng/μL (Med=91.2 ng/μL, 25^{th} percentile=36.6 ng/μL and 75^{th} percentile=117 ng/μL) and correlated with the initial DNA concentrations (Spearman r=0.443, P=0.003).

The concentrations of the final 16S libraries amplified from non-pretreated DA samples (i.e., those for which DTT was added only for the removal of the ALL PROTECT reagent) ranged from 0.01 ng/μl to 208 ng/μl (med=19.5 ng/μl, 25% percentile=3.3 ng/μl and 75% percentile=69.8 ng/μl) and correlated with the initial DNA concentration (Spearman r=0.228, p=0.027). Amplicons from non-pretreated DA libraries with low DNA concentrations showed less intense fragments on the Bioanalyzer gel and some had no fragments of the expected size (˜550 bp).

### Sequencing Results

All samples had at least 9,000 sequences and no exclusions were performed. A total of 112 DA-U and 43 DA-DTT samples were sequenced. The difference in average library sizes between the groups was less than 2-fold (Table 1). Predictions for significant differentially abundant Operational Taxonomic Units (OTUs) were performed following recommendations from McMurdie and Holmes, and from Weiss et al., used when the average library size for each group is approximately equal and/or the fold difference between groups is not high (>2-3× on average).

Considering observations regarding contamination of DNA extraction kits with traces of bacterial DNA,16S sequencing was also performed on negative control samples (DTT only). Less than 0.03% of the total sequences generated in each MiSeq run was assigned to negative control samples, 4,433 sequences on average. 27.63% of the sequences assigned to negative controls were identified as bacterial DNA, mostly belong to the Pseudomonas genus OTU 646549 (63.5%), and Bacteroides genus OTUs 1749079, 193591 and 359538 (12%).

All OTUs observed in negative controls were also detected in both groups analyzed, DA-U and DA-DTT. The OTUs assigned to Bacteroides genus observed in negative controls represented less than 3% of all OTUs assigned to this same genus in DA-U and DA-DTT, thus these OTUs were not excluded during downstream analysis. The OTU assigned to Pseudomonas genus (646549) observed in negative controls represented 67% of the OTUs assigned to this same genus in DA-U and DA-DTT, and considering the high risk of bias during analysis the OTU 646549 was excluded during comparisons between DA-U and DA-DTT groups.

TABLE 1
16S library size of DA-DTT and DA-U samples

DA - All subjects
(n = 155)

| | Non- | DTT- |
|---|---|---|
| Library size | pretreated | pretreated |
| (sequences) | (n = 112) | (n = 43) |
| Mean | 236,525 | 174,525^{a} |
| Standard Deviation | 157,694 | 92,366 |
| Standard Error of Mean | 14,901 | 14,086 |
| Median | 199,936 | 169,737^{a} |
| 25% Percentile | 118,973 | 101,216 |
| 75% Percentile | 329,521 | 199,936 |

^{a}Mann-Whitney test p < 0.05. Library size comparison between DTT-pretreated DA vs non-pretreated DA.
^{b}Mann-Whitney test p ≥ 0.05. Library size comparison between DTT-pretreated DA vs non-pretreated DA.

### DTT Increases the Detected Relative Abundance of Anaerobic Bacteria in DA

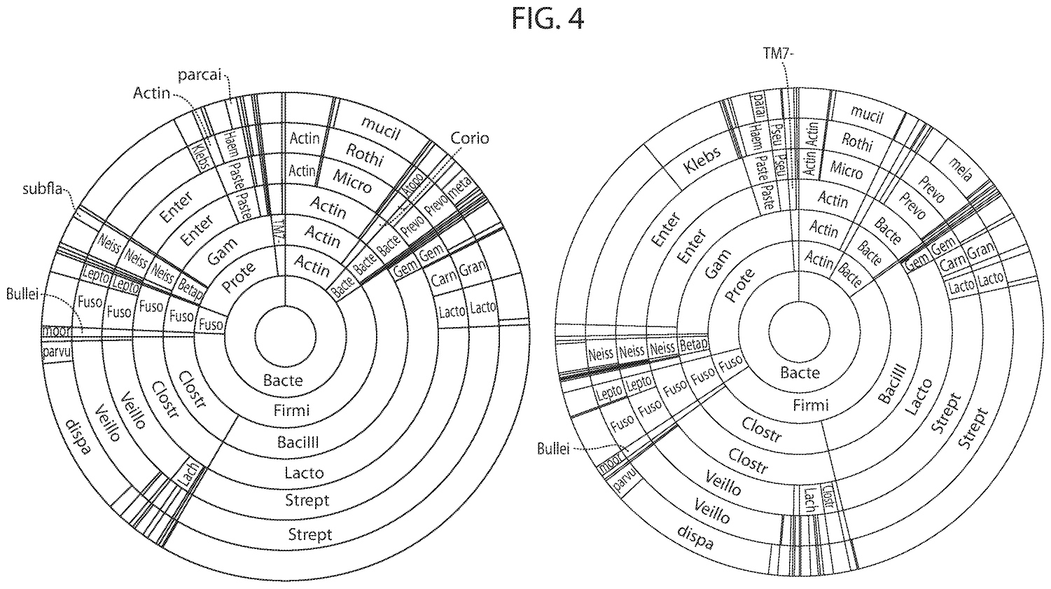

The main two dominant phyla observed in DA-DTT and DA-U were Firmicutes and Proteobacteria, followed by smaller proportions of Actinobacteria, Fusobacteria, Bacteroidetes and TM7 (FIG. 4, Table 2). After pretreatment with DTT, DA showed increased relative abundance of the phyla Proteobacteria (FC=6.22, FDR P=7.71E-7), Bacteroidetes (FC=2.19, FDR P=0.03) and Fusobacteria (FC=1.96, FDR P=0.03), when compared to DA-U (Table 2). There were also smaller changes in the relative abundances of Actinobacteria and TM7 that did not reach significance (Table 1).

TABLE 2
Differential abundance of the top six phyla in DA-DTT versus DA-U

| | DA-DTT (n = 43) versus DA-U (n = 112) | | | | |
|---|---|---|---|---|---|
| | Average | Average | Fold Change | | |
| | Relative | Relative | (calculated | | |
| | abundance % | abundance % | from the | P- | FDR P- |
| Taxonomy | DA-DTT^{#} | DA-U^{#} | GLM)* | value | value |
| Firmicutes | 49.3 | 62.25 | 1.05 | 0.65 | 0.70 |
| Proteobacteria | 28.97 | 14.8 | 6.22 | 1.4E−7 | 7.71E−7 |
| Actinobacteria | 8.91 | 12.02 | −1.23 | 0.21 | 0.42 |
| Fusobacteria | 5.36 | 3.93 | 1.96 | 0.01 | 0.03 |
| Bacteroidetes | 6.16 | 4.63 | 2.19 | 0.01 | 0.03 |
| TM7 | 1.17 | 1.86 | −1.34 | 0.32 | 0.48 |

P-value < 0.05 and FDR P-value < 0.05 are shown in bold.
^{#}The relative abundances were calculated from the original counts (number of sequences in the OTU table).
*Fold changes were calculated from the GLM, which corrects for differences in library size between the samples and the effects of confounding factors. It is therefore not possible to derive these fold changes from the original counts (number of sequences in the OTU table) by simple algebraic calculations.

Although no changes were seen in the detected relative abundances of Clostridia and Bacilli, the two main classes from the phylum Firmicutes, in DA-DTT vs. DA-U, specific increases were observed in families from both of these classes. Specifically, DA-DTT exhibited increased detected relative abundances of the family Clostridiaceae (FC=5.10, FDR P<0.0001) and genus Clostridium (FC=4.06, FDR P=4.38E-6), which are Gram-positive obligate anaerobes, and of the family Enterococcaceae (FC=76.22, FDR P=2.62E-11) and genus Enterococcus (FC=42.18, FDR P=2.57E-8), which are Gram-positive facultative anaerobic lactic acid bacteria (Table 3, FIG. 4).

The detected relative abundances of several obligate anaerobic bacteria were increased in DA-DTT vs. DA-U, including Fusobacterium (phylum Fusobacteria), which are Gram-negative bacilli (FC=2.29, FDR P=0.02), and Bacteroides (phylum Bacteroidetes) (FC=28.08, FDR P=5.43E-9) (Table 3, FIG. 4).

TABLE 3
Differential abundance of anaerobic bacteria in DA-DTT versus DA-U.

| | DA-DTT (n = 43) vs. DA-U (n = 112) | | | | |
|---|---|---|---|---|---|
| | Average | Average | Fold Change | | |
| | Relative | Relative | (calculated | | |
| | abundance % | abundance % | from the | | FDR |
| Taxonomy | DA-DTT^{#} | DA-U^{#} | GLM)* | P-value | P-value |
| p_Firmicutes, c_Clostridia, | 0.032 | 0.024 | 4.06 | 1.22E−6 | 4.38E−6 |
| f_Clostridiaceae, g_Clostridium | | | | | |
| p_Firmicutes, c_Bacilli, | 0.661 | 0.009 | 42.18 | 5.57E−9 | 2.57E−8 |
| f_Enterococcaceae, | | | | | |
| g_Enterococcus | | | | | |
| p_Fusobacteria, c_Fusobacteriia, | 3.625 | 2.471 | 2.29 | 0.01 | 0.02 |
| f_Fusobacteriaceae, | | | | | |
| g_Fusobacterium | | | | | |
| p_Bacteroidetes, c_Bacteroidia, | 0.626 | 0.073 | 28.08 | 1.08E−9 | 5.43E−9 |
| f_Bacteroidaceae, g_Bacteroides | | | | | |

P-value < 0.05 and FDR P-value < 0.05 are shown in bold.
^{#}The relative abundances were calculated from the original counts (number of sequences in the OTU table).
*Fold changes were calculated from the GLM, which corrects for differences in library size between the samples and the effects of confounding factors. It is therefore not possible to derive these fold changes from the original counts (number of sequences in the OTU table) by simple algebraic calculations.

### Pretreatment with DTT Increased the Detected Relative Abundance of Gram-Negative Enteropathogens from the Phylum Proteobacteria

The relative abundance of the phylum Proteobacteria, a major phylum of Gram-negative bacteria, detected in DA-DTT was increased compared to that detected in DA-U (FC=6.22, FDR P=7.71E-7). The detected relative abundances of three of the five most important classes from this phylum were significantly increased in DA-DTT compared to DA-U-class Gammaproteobacteria, which comprises several enteropathogens (FC=8.44, FDR P=4.25E-8), class Alphaproteobacteria, which includes mainly phototrophic bacteria (FC=7.94, FDR P=2.60E-8), and class Deltaproteobacteria, which includes sulfate- and sulfur-reducing bacteria (FC=6.35, FDR P=9.7E-5) (Table 4). Smaller changes in classes Betaproteobacteria and Epsilonproteobacteria did not reach significance (Table 4).

The increase in the detected relative abundance of Gammaproteobacteria in DA-DTT was partially driven by higher detected relative abundances of Enterobacteriaceae family members (FC=5.46, FDR P=1.47E-3), including important enteropathogens and pathogens that cause infection in several parts of the human body, such as Klebsiella and Providencia (see Table 5). The detected relative abundances of other members of the class Gammaproteobacteria were also increased in DA-DTT vs. DA-U, including the family Aeromonadaceae (FC=63.61, FDR P=1.18E-13) and the genus Pseudomonas (family Pseudomonadaceae) (FC=2.65, FDR P=0.04).

TABLE 4
Differential abundance of members of the phylum
Proteobacteria in DA-DTT versus DA-U

| | DA-DTT (n = 43) vs. DA-U (n = 112) | | | | |
|---|---|---|---|---|---|
| | | | Fold | | |
| | Average | Average | Change | | |
| | relative | relative | (calculated | | |
| | abundance % | abundance % | from the | | FDR P- |
| Taxonomy | DA-DTT | DA-U | GLM)* | P-value | value |
| p_Proteobacteria, | 23.823 | 10.492 | 8.44 | 8.3E−9 | 4.25E−8 |
| c_Gammaproteobacteria | | | | | |
| p_Proteobacteria, | 1.294 | 0.145 | 7.94 | 4.05E−9 | 2.60E−8 |
| c_Alphaproteobacteria | | | | | |
| p_Proteobacteria, | 0.008 | 0.001 | 6.35 | 3.08E−5 | 9.70E−5 |
| c_Deltaproteobacteria | | | | | |
| p_Proteobacteria, | 3.569 | 4.029 | −1.26 | 0.41 | 0.56 |
| c_Betaproteobacteria | | | | | |
| p_Proteobacteria, | 0.281 | 0.167 | 1.74 | 0.14 | 0.29 |
| c_Epsilonproteobacteria | | | | | |

P-value < 0.05 and FDR P-value < 0.05 are shown in bold.
^{#}The relative abundances were calculated from the original counts (number of sequences in the OTU table).
*Fold changes were calculated from the GLM, which corrects for differences in library size between the samples and the effects of confounding factors. It is therefore not possible to derive these fold changes from the original counts (number of sequences in the OTU table) by simple algebraic calculations.

TABLE 5
Differential abundance of members of the family
Enterobacteriaceae in DA-DTT versus DA-U.

| | DA-DTT (n = 43) vs. DA-U (n = 112) | | | | |
|---|---|---|---|---|---|
| | | | Fold | | |
| | Average | Average | Change | | |
| | relative | relative | (calculated | | |
| | abundance % | abundance % | from the | | FDR P- |
| Taxonomy | DA-DTT | DA-U | GLM)* | P-value | value |
| c_Gammaproteobacteria, | 19.193 | 6.068 | 5.46 | 5.13E−4 | 1.47E−3 |
| o_Enterobacteriales, | | | | | |
| f_Enterobacteriaceae | | | | | |
| f_Enterobacteriaceae, | 14.984 | 5.227 | 17.00 | 2.72E−8 | 1.21E−7 |
| g_unknown | | | | | |
| f_Enterobacteriaceae, | 3.812 | 0.784 | 24.10 | 7.13E−7 | 2.73E−6 |
| g_Klebsiella | | | | | |
| f_Enterobacteriaceae, | 0.224 | 0.00025 | 13.57 | <0.0001 | <0.0001 |
| g_Providencia | | | | | |
| f_Enterobacteriaceae, | 0.018 | 0.006 | 36.71 | 1.18E−9 | 5.81E−9 |
| g_Morganella | | | | | |
| f_Enterobacteriaceae, | 0.006 | 0.001 | 3.71 | 0.01 | 0.02 |
| g_Salmonella | | | | | |

P-value < 0.05 and FDR P-value < 0.05 are shown in bold.
^{#}The relative abundances were calculated from the original counts (number of sequences in the OTU table).
*Fold changes were calculated from the GLM, which corrects for differences in library size between the samples and the effects of confounding factors. It is therefore not possible to derive these fold changes from the original counts (number of sequences in the OTU table) by simple algebraic calculations.

The increase in relative abundances of Alphaproteobacteria and Deltaproteobacteria in DTT-pretreated DA was driven by increases in the order Rhizobiales (FC=19.03, FDR p=4.33E-13), and in sulfur-producing bacteria from the orders Desulfobacterales (FC=42.61, FDR p<0.0001) and Desulfovibrionales (FC=6.41, FDR p=6.71E-3), respectively.

### Predicted Metabolic Functions in DTT-Pretreated DA

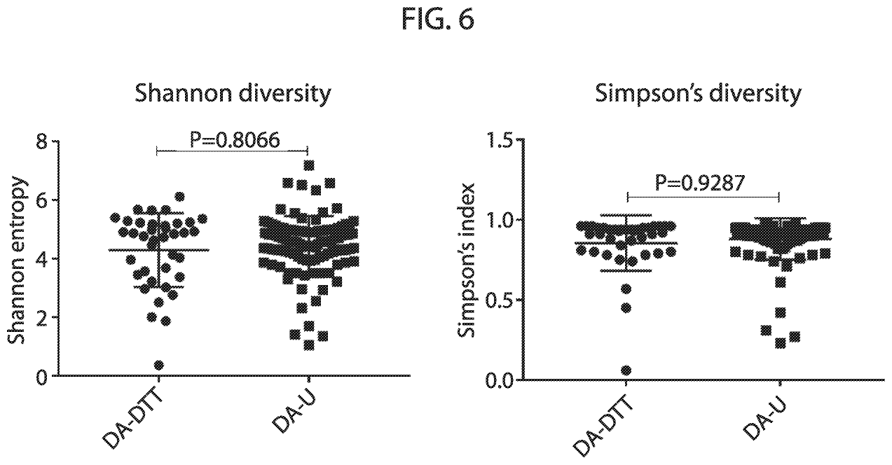

The microbiome-associate-predicted metabolic functions of DTT-pretreated DA showed several differences when compared to non-pretreated DA (FIG. 6). The KEGG level 2 results of DTT-pretreated DA displayed high number of sequences assigned to glycan biosynthesis and metabolism, lipid metabolism, such as steroid hormone biosynthesis, and cell motility, such as flagellar assemble and bacterial motility proteins. The mean NSTI value was 0.053 for non-pretreated DA and 0.046 for DTT-pretreated DA, which indicate that the predicted metabolic functions displayed by the observed microbial community in both groups are close to the known microbial reference genome databases, and thus imply a higher accuracy of the predictions.

### Pretreatment with DTT Maintains Microbial Diversity in DA

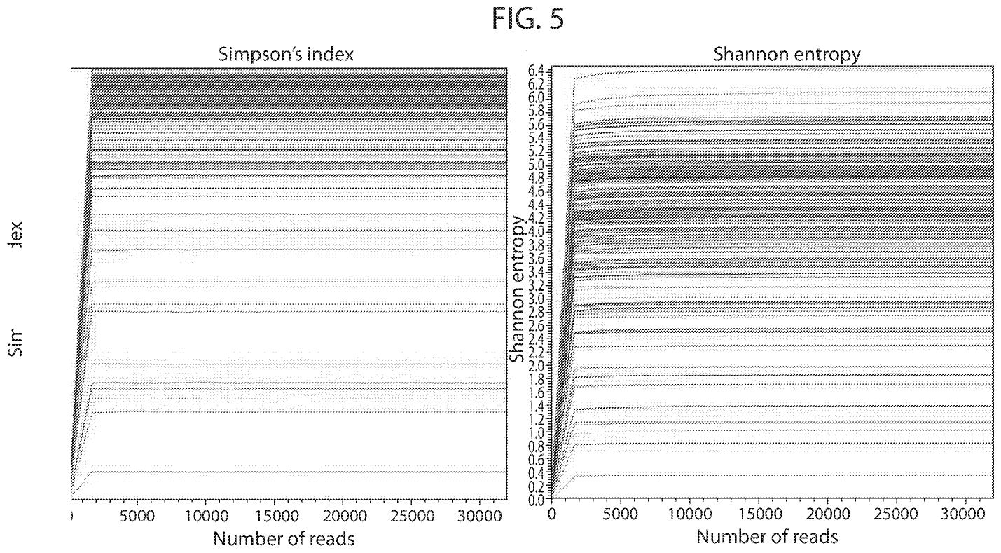

Sample rarefaction curves showed a similar pattern, which verified that most of the species present in each sample from DA-DTT and DA-U groups were observed (FIG. 5). DA-DTT exhibited the same alpha diversity as DA-U, as demonstrated by Simpson's index (P=0.9287) and Shannon entropy (P=0.8066) (FIG. 6).

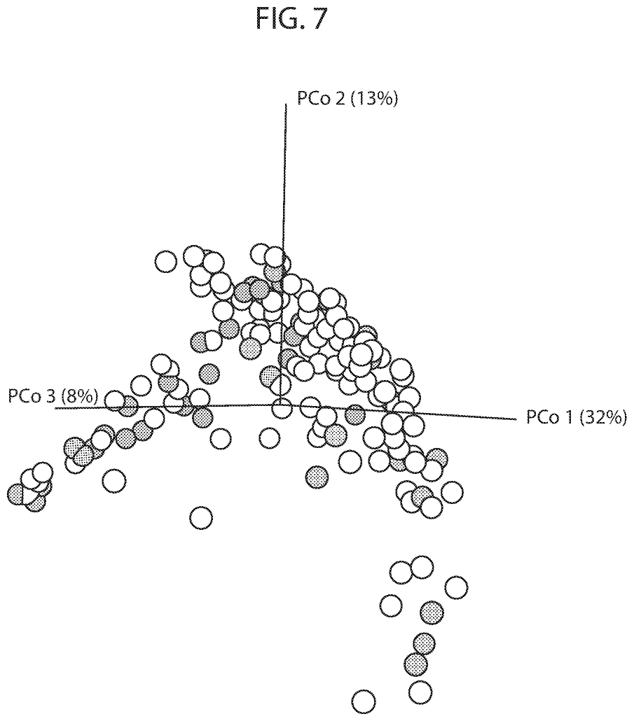

Beta diversity of the DA-U and DA-DTT microbiome was analyzed based on the weighted UniFrac metric. Principal Coordinate Analysis plot showed no clustering of the DA-DTT (n=43) and DA-U groups (n=112) (see FIG. 7).

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method of increasing the number of microorganisms extracted from a biological sample, comprising:
adding a reducing agent to the biological sample to produce a mixture; and
centrifuging the mixture to produce a supernatant and a pellet,
wherein the number of microorganisms extracted is greater than the number of microorganisms extracted without the addition of a reducing agent.

2. The method of claim 1, wherein the biological sample is a viscous biological sample.

3. The method of claim 1, wherein the biological sample was stored with a preservation and/or stabilization reagent.

4. The method of claim 1, wherein the reducing agent is selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa, and combinations thereof.

5. The method of claim 1, wherein the reducing agent is dithiothreitol (DTT).

6. The method of claim 1, wherein the reducing agent is N-acetylcysteine (NAC)

7. The method of claim 1, further comprising conserving the pellet in a preservation and/or stabilization reagent.

8. The method of claim 7, wherein the preservation and/or stabilization reagent is Allprotect® reagent.

9. The method of claim 1, wherein the viscosity of the biological sample is reduced.

10. The method of claim 1, wherein the biological sample is selected from the group consisting of large or small intestinal fluids or aspirate, stomach fluids or aspirate, mucus, mucous membrane, stool, whole blood, plasma, serum, cerebral spinal fluid (CSF), urine, sweat, saliva, tears, pulmonary secretions, breast aspirate, prostate fluid, seminal fluid, cervical scraping, vaginal secretions, amniotic fluid, semen, synovial fluid, intraocular fluid, moisture in breath, sputum, liquid sample from inflamed tissue, and combinations thereof.

11. The method of claim 1, wherein the biological sample is selected from the group consisting of intestinal biopsies, small or large intestinal fluids or aspirates, and combinations thereof.

12. The method of claim 11, wherein the small intestinal fluids or aspirates is duodenal fluids or aspirate.

13. The method of claim 1, wherein the biological sample is selected from the group consisting of urine, saliva, sputum, liquid samples from inflamed tissue, mucous membranes, amniotic fluid, vaginal secretions, semen, synovial fluid, stool.

14. The method of claim 1, further comprising performing DNA extraction, DNA library preparation, DNA quantification, and/or DNA sequencing on the pellet.

15. A method, comprising:
adding a reducing agent to a biological sample or biological sample that was stored with a preservation and/or stabilization reagent to produce a mixture; and
centrifuging the mixture to produce a supernatant and a pellet.

16. The method of claim 15, wherein the reducing agent is selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa, and combinations thereof.

17. The method of claim 15, wherein the reducing agent is dithiothreitol (DTT).

18. The method of claim 15, wherein the reducing agent is N-acetylcysteine (NAC).

19. The method of claim 15, further comprising conserving the pellet in a preservation and/or stabilization reagent.

20. The method of claim 19, wherein the preservation and/or stabilization reagent is Allprotect® reagent.

21. The method of claim 15, wherein the viscosity of the biological sample is reduced.

22. The method of claim 15, wherein the biological sample is selected from the group consisting of large or small intestinal fluids or aspirate, stomach fluids or aspirate, mucus, mucous membrane, stool, whole blood, plasma, serum, cerebral spinal fluid (CSF), urine, sweat, saliva, tears, pulmonary secretions, breast aspirate, prostate fluid, seminal fluid, cervical scraping, vaginal secretions, amniotic fluid, semen, synovial fluid, intraocular fluid, moisture in breath, sputum, liquid sample from inflamed tissue, and combinations thereof.

23. The method of claim 15, wherein the biological sample is selected from the group consisting of intestinal biopsies, small or large intestinal fluids or aspirates, and combinations thereof.

24. The method of claim 23, wherein the small intestinal fluids or aspirates is duodenal fluids or aspirate.

25. The method of claim 15, wherein the biological sample is selected from the group consisting of urine, saliva, sputum, liquid samples from inflamed tissue, mucous membranes, amniotic fluid, vaginal secretions, semen, synovial fluid, stool.

26. The method of claim 15, further comprising performing DNA extraction, DNA library preparation, DNA quantification, and/or DNA sequencing on the pellet.

27-31. (canceled)

32. The method of claim 15, wherein the preservation and/or stabilization reagent is Allprotect® reagent.

33-37. (canceled)

38. A kit, comprising:
a quantity of a reducing agent; and
instructions for using the quantity of the reducing agent to reduce the viscosity of a liquid sample.

39. The kit of claim 38, further comprising a preservation and/or stabilization reagent.

40. The kit of claim 38, wherein the reducing agent is selected from the group consisting of 2-Mercaptoethanol, 2-Mercaptoethylamine-HCl, Bond-Breaker TCEP Solution, NeutralpH, Cysteine-HCl, TCEP-HCl, TCEP-HCl (Premium Grade), Immobilized Reductant Columns, Immobilized TCEP Disulfide Reducing Gel, 8M Guanidine-HCl Solution, Guanidine-HCl, Urea, N-acetylcysteine (NAC), acetylcysteine, ambroxol, bromhexine, carbocisteine, erdosteine, mecysteine, dornase alfa, dithiothreitol (DTT), and combinations thereof.

41. (canceled)

42. (canceled)


SEQUENCE LISTING (2 sequences)

SEQ ID NO: 1 - DNA, 50 bp, Artificial Sequence
  synthetic construct
  misc_feature (42)..(42): n is a, c, g, or t
tcgtcggcagcgtcagatgtgtataagagacagcctacgggnggcwgcag

SEQ ID NO: 2 - DNA, 55 bp, Artificial Sequence
  synthetic construct
  misc_feature (41)..(41): h is a, c, or t
  misc_feature (42)..(42): v is a, c, or g
gtctcgtgggctcggagatgtgtataagagacaggactachvgggtatctaatcc